(19) United States
(12) Patent Application Publication
(10) Pub. No.: US 2016/0188823 A1
(43) Pub. Date: Jun. 30, 2016

(54) SYSTEM FOR PROVIDING ELECTROCARDIOGRAM (ECG) ANALYTICS FOR ELECTRONIC MEDICAL RECORDS (EMR)

(71) Applicant: General Electric Company, Schenectady, NY (US)

(72) Inventors: Gordon Ian Rowlandson, Wauwatosa, WI (US); Brian J. Young, Wauwatosa, WI (US); Mark Gilbert Langer, Mequon, WI (US)

(73) Assignee: GENERAL ELECTRIC COMPANY, Schenectady, NY (US)

(21) Appl. No.: 14/587,607

(22) Filed: Dec. 31, 2014

(51) Int. Cl. G06F 19/00; A61B 5/044; A61B 5/04

(52) U.S. Cl. CPC G06F 19/345; A61B 5/04012; A61B 5/044; G06F 19/322; G06F 19/3487

(57) ABSTRACT

The system of the present application includes computerized diagnostic ECG that is tailored for the EMR. The system and method of the present application provides several new approaches to the computerized ECG based on information available from the EMR through an EMR portal. Some of these information items include: Test indication and reason for performing the ECG, previous ECGs as a measure of the patient's “normal” baseline; electrolytes, and drugs known to cause cardiac toxicity/prolonged QT. Based on these inputs, the computerized ECG analysis will behave differently, including the formation of reports, the ancillary information supplied with the ECG and the interpretation itself.

### FIELD

The present disclosure generally relates to electrocardiogram (ECG) analysis. More specifically, the present disclosure relates to integrating ECG analytics with electronic medical records (EMR).

### BACKGROUND

In current systems, EMRs simply place the image of an ECG report into a patient record as part of a list of other tests. Sometimes only the interpretation is provided without an image. Furthermore, there is no interaction with the ECG image nor is there any optimization of the analysis based on what is available via the EMR. Also, due to the advent of the EMR, the editor of the ECG is migrating from the cardiologist to the practitioner—the one who is actually making care decisions based on the ECG.

The format of the computerized ECG has not changed since it was introduced over 30 years ago. This is despite the fact that reasons for doing an ECG have changed as well as its relative value versus other diagnostic tools now available, such as electrocardiography, nuclear imaging, and other. Nevertheless, any change in the format of the ECG cannot be too provocative since it is universally recognized as an ECG: and interpretable in its current form across the world. Moreover, most electro-cardiographers believe they can, in a matter of seconds, holistically recognize a particular ECG as being related to a specific diagnostic finding. As a result, anything that interferes with that process of recognition should be considered off limits. It is desirable to speed up the process and make the ECG report more useful for both the prescribing, physician and over-reading, cardiologist. In any case, given the adoption of the EMR, the availability of ancillary information such as lab results in the EMR and other additional information, the advent of inexpensive as well as portable diagnostic imaging devices, the increased focus on cost-containment and the diminishing reimbursement for the cardiologist in editing the ECG—the computerized ECG needs to be updated.

Attempts to include such information as patient history in the computerized ECG have been attempted using the electrocardiograph. However, information entered at the electrocardiograph is notoriously inaccurate. Age or patient name information is incorrect 15% of the time. Thus, most computerized ECG programs do not rely heavily on this information otherwise the program would be prone to error.

Referring to FIG. 1, existing ECG reports 100 evolved out of a 3 or 6 channel ink writer, equipped with a dot matrix printer that could only supply alphanumeric information at the top of the report 100. The ECG report 100 example in FIG. 1 illustrates a 6-channel ECG report 100, the 6-channel ECG 105 illustrating a number of ECGs taken from a single patient. The ECG report 100 also includes patient information and ECG data 110 above the 6-channel ECG 105. Thus, current systems are very similar to what existed 30 years ago: that is, black and white characters at the top of the ECG report 100 with black waveforms superimposed over a pre-printed grid. Ironically, all current systems continue to present information in this manner, even if it is being supplied as part of an EMR.

### SUMMARY

The system of the present application includes computerized diagnostic ECG that is tailored for the EMR. The system and method of the present application provides several new approaches to the computerized ECG based on information available from the EMR through an EMR portal. Some of these information items include: Test indication and reason for performing the ECG, previous ECGs as a measure of the patient's “normal” baseline; electrolytes, and drugs known to cause cardiac toxicity/prolonged QT. Based on these inputs, the computerized ECG analysis will behave differently, including the formation of reports, the ancillary information supplied with the ECG and the interpretation itself. Furthermore, quality control procedures when associating clinical data with a patient record within an EMR system ensures the accuracy and quality of data that is included with the ECG that is required to obtain accurate computerized analysis results and overcome limitations of prior attempts to do this at the electrocardiograph.

In one embodiment of the present application, A system for electrocardiogram (ECG) analysis, comprising: a processor configured to receive ECG data and electronic medical record (EMR) through an EMR portal, data potentially correlated with the ECG data, and to analyze the ECG data and EMR data, and a display operatively connected to the processor and configured to convey an ECG report comprising the EMR data.

In another embodiment of the present application, a graphical user interface (GUI) including a display and an input device as a method for providing, electrocardiogram (ECG) data in an electronic medical record (EMR), the method including receiving ECG data and electronic medical record (EMR) data, correlating the EMR data with the ECG data, analyzing the ECG data and EMR data, and conveying an ECG report in the GUI, wherein the ECG report includes the EMR data and the ECG data.

In another embodiment of the present application, A system for electrocardiogram (ECG) analysis, including, an ECG monitor configured to collect ECG data from a patient, an electronic medical record (EMR) database configured to store EMR data for the patient, a processor configured to receive the EMR data, and retrieve the ECG data from the ECG monitor through a portal, wherein the EMR data is correlated with the ECG data, and the processor is further configured to analyze the ECG data and EMR data, and a display operatively connected to the processor and configured to convey an ECG report in a graphical user interface, wherein the ECG report includes the EMR data and the ECG data.

### BRIEF DESCRIPTION OF THE DRAWINGS

FIG. 1 is a graphical representation of a graphical user interface (GUI) of the prior art;

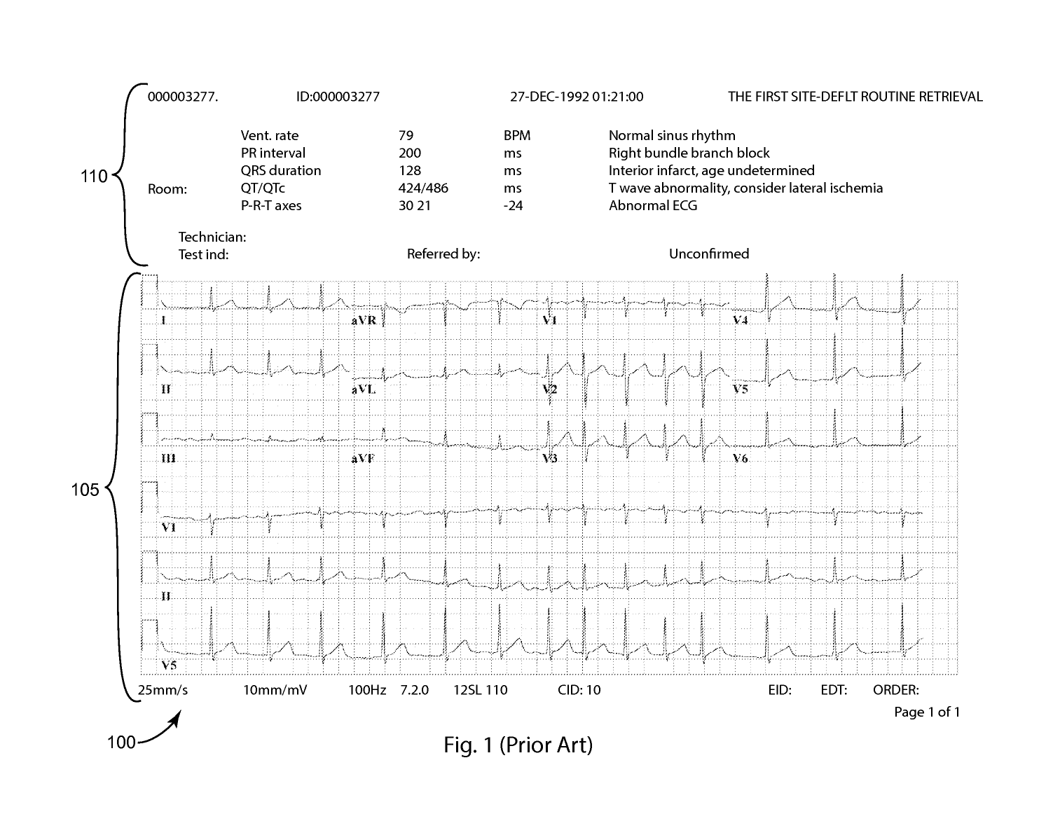

FIG. 2 is a graphical representation of a GUI according to an embodiment of the present application;

FIG. 3 is a graphical representation of a GUI according to an embodiment of the present application;

FIG. 4 is a graphical representation of a GUI according to an embodiment of the present application; and

FIG. 5 is a graphical representation of a GUI according to an embodiment of the present application.

FIG. 6 is a system diagram illustrating an ECG/EMR system according to an embodiment of the present application.

FIG. 7 is a system diagram illustrating an ECG/EMR system according to an embodiment of the present application.

### DETAILED DESCRIPTION

In the present description, certain terms have been used for brevity, clearness and understanding. No unnecessary limitations are to be applied therefrom beyond the requirement of the prior art because such terms are used for descriptive purposes only and are intended to be broadly construed. The different systems and methods described herein may be used alone or in combination with other systems and methods. Various equivalents, alternatives and modifications are possible within the scope of the appended claims. Each limitation in the appended claims is intended to invoke interpretation under 35 U.S.C., §112, sixth paragraph, only if the terms “means for” or “step for” are explicitly recited in the respective limitation.

In the following detailed description, reference is made to the accompanying drawings that form a part hereof, and in which is shown by way of illustration specific embodiments that may he practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

The system and method of the present application improves the operation of the current ECG and EMR systems in a number of ways: human over-reading is improved when the over-reader is aware of a patient's condition—the EMR coupled with the computerized ECG enables this; also in general, ECG interpretation should be guided by the reason for the test which would now be reliably available from EMR; and if serial ECGs are the same in current systems, then the current systems will consistently make the same mistake. Instead of correcting each one of these, the system and method of the present application may pull forward the previous edited and/or corrected interpretation onto the current report from an EMR portal; certain drugs and electrolyte values impact how the ECG should be interpreted; by interacting with the report through the EMR portal, the report is not cluttered, but instead provides information when values are not within normal bounds; and ECGs acted upon as part of care decisions can be edited by the prescribing physician within the EMR.

The technical effect and advantage is that the computerized ECG will be more accurate and tailored for the reason it was obtained. Again, the system and method of the present application improves the operation of ECG monitoring and EMR practice by making current systems more reliable and accurate. The current systems are also improved in that every prescribing physician uses the ECG but sonic actually do not understand the implications of the test. If the test is tailored to the reason why the prescribing physician ordered it, then it will be more valued by the physician. EMRs are becoming the center of data entry, and departmental systems are being replaced by inferior EMR applications that present the ECG. By providing a superior solution that can plug, into the EMR through the EMR portal, both the EMR and the ECG are improved.

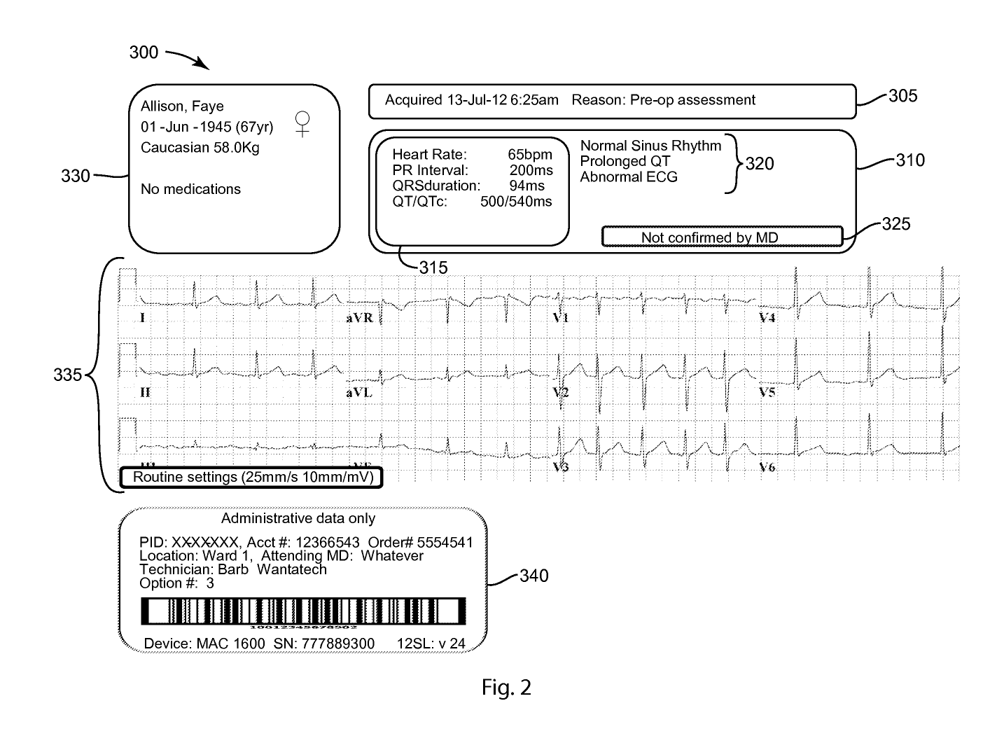

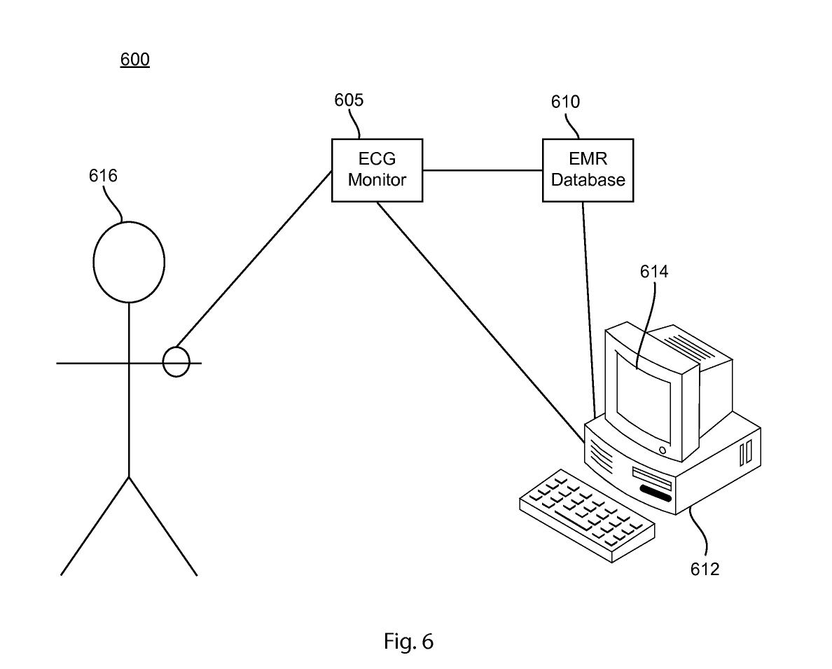

FIG. 2 is an example of an ECG report 300 of the present application. Referring to FIG. 2 and FIG. 6 simultaneously, this ECG report 300 and the subsequent reports discussed herein are displayed for a user on a graphical user interface (GUI) 614. The reason for the test on the test indication 305 is obtained from the EMR database 610 for the monitored patient 616. The ECG data values 315 illustrate in quantitative values what is shown in the ECG data 335. Any of these data values 315 may be highlighted in a variety of ways (e.g., different color, different text, underline, bold) to alert a user of an abnormal value. Current electrocardiograph systems do not highlight abnormal values as such. The ECG report 300 includes a clinical findings section 310 that may include a number of tools, including the ECG data values section 315, and ECG results section 320, and a MD confirmation button 325. It is contemplated that any of these sections and/or buttons may be “turned off” by a user using the user interface 210 so that the ECG report 300 is configurable by the user. The position and size of each of these sections may also be configurable by the user. It is also possible for a user to set the ECG report 300 such that values in the ECG data values section 315 that are abnormal may be highlighted, but without comment in the ECG results section 320 as to the meaning of the deviation or abnormality. In the example of FIG. 2, if the QT/QTC value is highlighted and therefore abnormal, then perhaps the “prolonged QT” result may be removed from the ECG report 300. This would be helpful for addressing a specific measurement such as QT, which has become something like a “disease” when you say it is prolonged when really, for some instances, a user may merely want to identify that the measurement is abnormal but no clinical conclusion should be made based on this information.

Still referring to FIG. 2 and FIG. 6 simultaneously, the ECG report 300 further includes a patient demographics section 330 that may include such patient information as name, birthdate, age, sex, race, weight, medication history, or any other standard patient information that is included in the EMR. As discussed previously, the test indication 305 allows the user to see the reason why the ECG is being obtained, and the time and date of such acquisition of the ECG. The confirmation button 325 alerts the user whether the data values and the results have been confirmed by the attending physician. Of course, the actual ECG data 335 is included in the ECG report 300. The ECG monitor settings include gain and filter settings for the ECG monitor 605. In current systems, the ECG monitor settings are oftentimes ignored, and it is contemplated that the system and method of the present application may highlight or flag this ECG monitor setting button 345 if the settings are outside of a predetermined range for both gain and filter. Last, the administrative record section 340 provides information in a barcode or other known scan and identification methods that allows the record to he located, as well as provide information regarding who created the record.

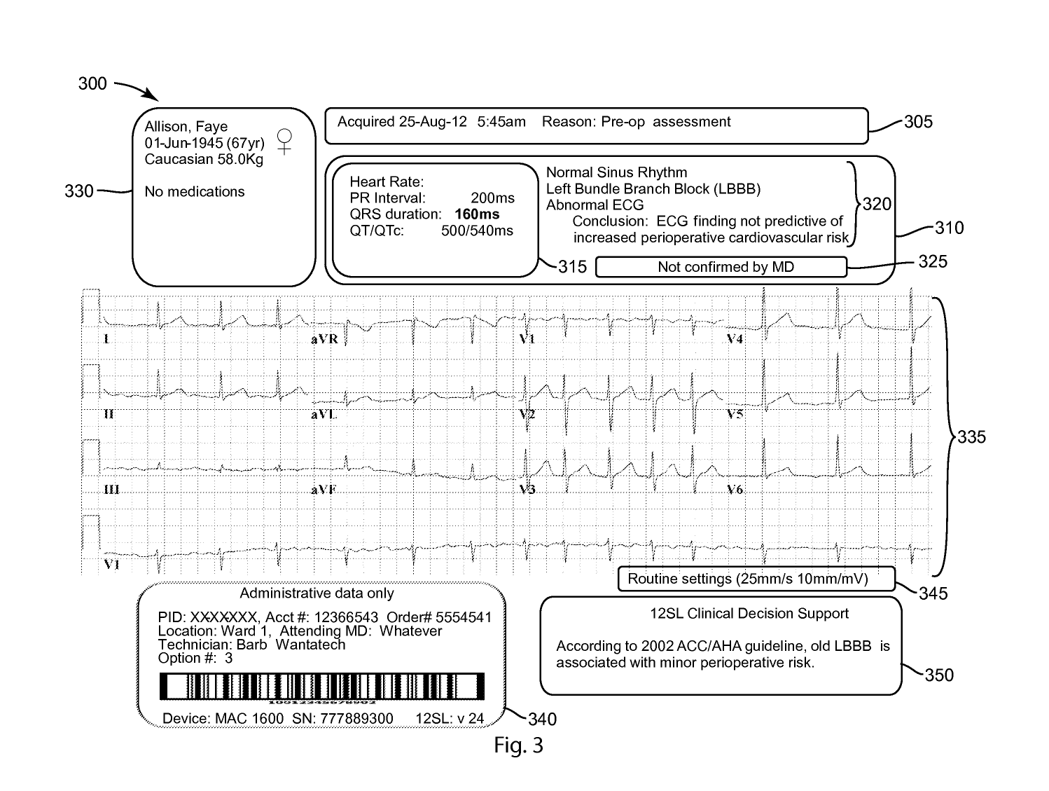

Referring now to FIG. 3 and FIG. 6, typically there are two physicians who use these types of ECG reports 300: the prescribing MD and the over-reading cardiologist. The prescribing MD may ask for the ECG data 335 to be taken for a specific purpose. The cardiologist provides a traditional over-read of the ECG data 335 but does not necessarily address the specific reason why the test was done. Therefore, in this embodiment of the present application, there are two areas for a clinical report: a conventional reading in the clinical findings section 310, and one that addresses the specific reason why the test may have been performed. At the bottom right of the ECG report 300 in FIG. 3 is another set of text in the Clinical Decision Support Box 350 that directly answers the reason for performing the ECG test, or in other words, a conclusion on the cardiac condition of the patient. The Clinical Findings Section Box 310 may include the standard ECG interpretation, while the Clinical Decision Support Box 350 directly answers the question: “Can this patient undergo surgery?”

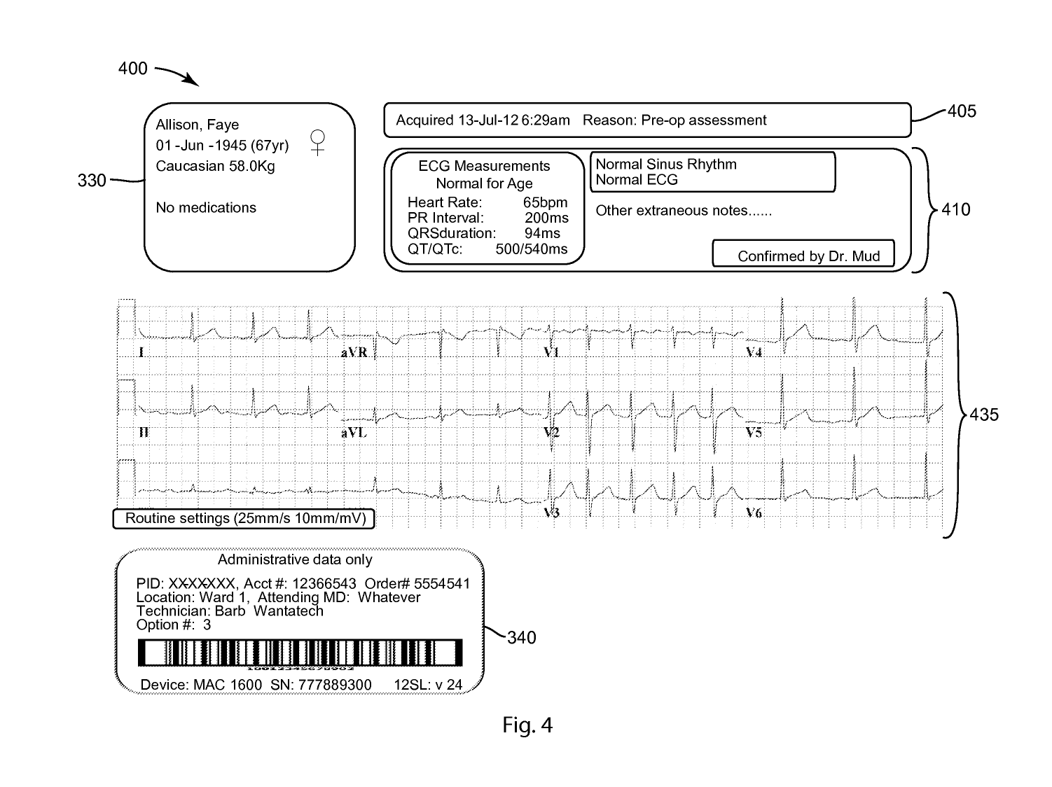

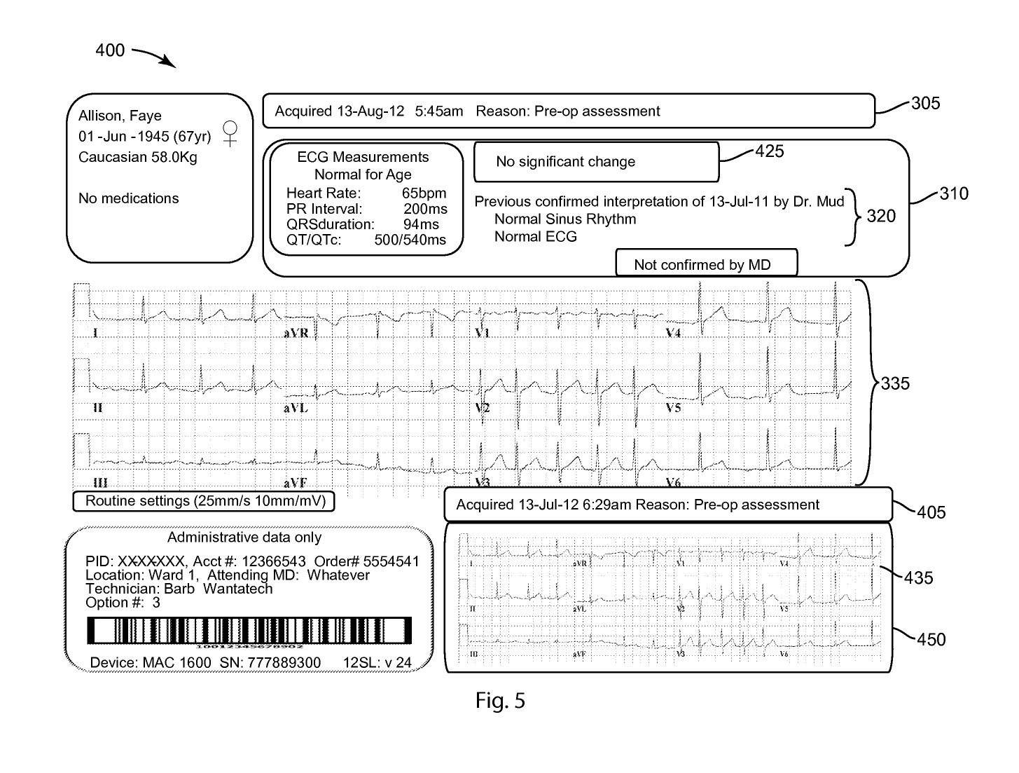

Referring now to FIG. 4 and FIG. 5, the next concept that is supported by the system of the present application is to illustrate to the user of the ECG the patient's baseline normal. Current systems have serial comparison capabilities, but do not provide, a user with the appropriate tools to evaluate the serial ECG to current ECG data.

Still referring to FIGS. 4 and 5, an ECG serial report 400 is illustrated. First referring to FIG. 4, a user may select a previous set of ECG data 435 to view in the ECG serial report 400. This previous ECG data 435 will also be accompanied by a test indication 405 and clinical findings section 410 just as at the time it was collected as described in FIG. 2. This ECG data 435 may then be compared to current ECG data 335 as illustrated in FIG. 5. Here, in the ECG serial report 400, the ECG serial data 435 is included in an ECG serial box 450 that further includes the test indication 405, identifying the time and reason for the collection of this ECG data 435. Furthermore, still referring to FIG. 5, the ECG results box 320 will include a status of the ECG serial data 435 and also a change statement 425 regarding the difference in interpretation from the ECG data 335 and the ECG serial data 435. It should be further noted that in any of these embodiments described above, the ECG reports 300, 400 may be configured in various colors and the shapes, position and presentations of the boxes may be altered slightly to accommodate display configurations, and to accommodate user specification.

In this embodiment, given that the change status 425 is at the top of the ECG serial report 400, the system may also pull forward the previous ECG results 320 that were confirmed by the MD. In order to do this, system must create a space on the ECG serial reports 400 that is reserved for the ECG report 300 that an MD would like to use when the ECG report 300 is pulled forward. In this case, the ECG serial box 450 is this space for previous ECG reports 300 and the ECG serial data 435.

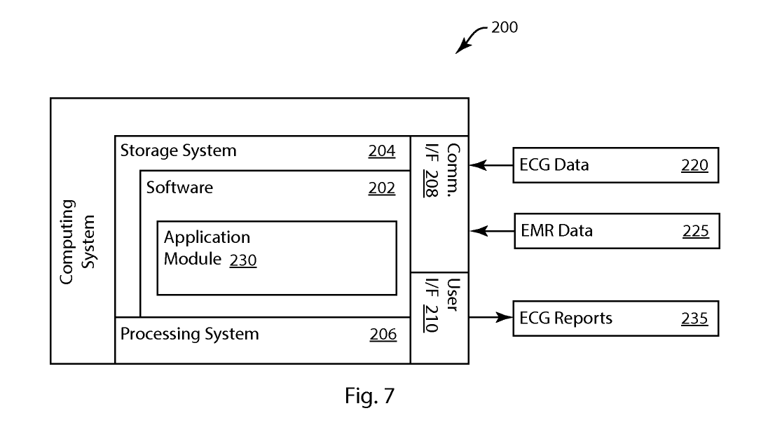

Referring now to FIG. 6, a system 600 of the present application is illustrated. Here, a patient 616 is being monitored by an ECG monitor 605. It should be understood that the ECG monitor 605 can be any monitor known in the art, portable or otherwise, that includes capabilities for collecting ECG data from the patient 616 and displaying the same. Furthermore, the ECG monitor 605 is configured to communicate with an EMR database 610. The EMR database 610 in the system 600 is also one known in the art. The computing system 612 includes a graphical user interface 614 that is capable of displaying any of the graphical user interfaces described in the previous figures. The computing system 612 is in communication with both the ECG monitor 605 and the EMR database 610. It should be further noted that the computing device 612 and the graphical user interface (GUI) 614 may be included within either of the ECG monitor 605 or the EMR database 610, in further embodiments. In other words, the computing device 612, which will be described in greater detail in FIG. 7, may be implemented in either one of the other two components of the system 600, namely the ECG monitor 605 or the EMR database 610. In one embodiment, a user accesses the GUI 614 on the computing device 612 through an application that accesses the EMR database 610. In other words, the GUI: 614 is a representation of an EMR of the patient 616 accessed from the EMR database 610. The EMR database 610 pulls ECG data from the ECG monitor 605 through an EMR portal. It is contemplated that other embodiments could operate in reverse, that is, the GUI 614 being a representation of a patient 616 ECG data accessed from the ECG monitor 605, the ECG monitor accessing EMR data for the patient 616 through a portal as well.

FIG. 7 is a system diagram of an exemplary embodiment of a computing system 200 for providing ECG analysis for EMR. The computing system 200 generally includes a processing system 206, storage system 204, software 202, communication interface 208 and a user interface 210. The processing system 206 loads and executes software 202 from the storage system 204, including a software module 230. When executed by the computing system 200, software module 230 directs the processing system 206 to operate as described herein in further detail in accordance with the system described in FIG. 6.

Although the computing system 200 as depicted in FIG. 7 includes one software module in the present example, it should be understood that one or more modules could provide the same operation. Similarly, while the description as provided herein refers to a computing system 200 and a processing, system 206, it is to be recognized that implementations of such systems can be performed using, one or more processors, which may be communicatively connected, and such implementations are considered to be within the scope of the description.

The processing, system 206 can comprise a microprocessor and other circuitry that retrieves and executes software 202 from storage system 204. The processing system 206 can be implemented within a single processing device but can also be distributed across multiple processing devices or sub-systems that cooperate in executing program instructions. Examples of processing system 206 include general-purpose central processing units, application specific processors, and logic devices, as well as any other type of processing device, combinations of processing devices, or variations thereof.

The storage system 204 can comprise any storage medium readable by processing system 206, and capable of storing software 202. The storage system 204 can include volatile and nonvolatile, removable and non-removable media implemented in any method or technology for storage of information, such as computer readable instructions, data structures, program modules, or other data. Storage system 204 can be implemented as a single storage device but may also be implemented across multiple storage devices or subsystems. Storage system 204 can further include additional elements, such as a controller capable of communicating with the processing system 206.

Examples of storage media include a random access memory, read only memory, magnetic disks, optical disks, flash memory, virtual memory, and non-virtual memory, magnetic sets, magnetic tape, magnetic disk storage or other magnetic storage devices, or any other medium which can be used to store the desired information and that may be accessed by an instruction execution system, as well as any combination or variation thereof, or any other type of storage medium.

User interface 210 can include a mouse, a keyboard, a voice input device, a touch input device for receiving a gesture from a user, a motion input device for detecting non-touch gestures and other motions by a user, and other comparable input devices and associated processing elements capable of receiving user input from a user. Output devices such as a video display or graphical display can display and interface further associated with embodiments of the system and method as disclosed herein. The speakers, printers, haptic devices, and other types of output devices may also be included in the user interface 210.

The user interface 210, as described above, may be located on the computing system 200, 612, or on either of the ECG monitors 605 or EMR database 610. The user interface 210 includes the graphical user interface 614, and allows the user to set the parameters for each of the ECG reports 300, 400 that the user requests the system to create, and further receives those ECG reports 235 from the user interface 210.

As described in further detail herein, the computing system 200 receives ECG data 220 and EMR data 225 at the communication interface 208. The ECG data 220 and the EMR data 225 is then processed by the processing system 206 in order to create the ECG reports 235 as described in detail previously in this application. The ECG Reports 235 are then outputted by the user interface 210 through the GUI 614 display in a computing device 612, or any other device capable of supporting the GUI 614 as further described in this application. In embodiments, the communication interface 208 operates to receive the ECG and EMR data 220, 225 from other devices to which the computing system 200 is communicatively connected. Such devices include, but are not limited to, the ECG monitor 605 and EMR database 610 of FIG. 6. As discussed above, the computing system 200 may receive the EMR data 225 from the EMR database 610, and pull the ECG data 220 through a portal from the EMR database 610. It should be understood that the ECG data 220 may include any ECG data collected from a 12-lead ECG device from a patient. The EMR data 225 can be any information typically included in the patient's electronic medical record.

In the foregoing description, certain terms have been used for brevity, clearness, and understanding. No unnecessary limitations are to be inferred therefrom beyond the requirement of the prior art because such terms are used for descriptive purposes and are intended to be broadly construed. The different configurations, systems, and method steps described herein may be used alone or in combination with other configurations, systems and method steps. It is to be expected that various equivalents, alternatives and modifications are possible within the scope of the appended claims.

I claim:

1. A system for electrocardiogram (ECG) analysis, comprising:
a processor configured to receive ECG data and electronic medical record (EMR) data, wherein the EMR data is correlated with the ECG data, and the processor is further configured to analyze the ECG data and EMR data; and
a display operatively connected to the processor and configured to convey an ECG report in a graphical user interface, wherein the ECG report includes the EMR data and the ECG data.

2. The system of claim 1, wherein the ECG report includes a test indication, wherein the test includes a time and a reason for an ECG test.

3. The system of claim 1, wherein the ECG report includes a clinical findings area.

4. The system of claim 3, wherein the clinical findings area of the ECG report further includes an ECG data values section, further wherein the ECG data values provides an indication of any abnormal values in the ECG data.

5. The system of claim 3, wherein the clinical findings area of the ECG report further includes an ECG results box, further wherein the ECG results box provide a standard ECG interpretation from the ECG data.

6. The system of claim 5, wherein the clinical findings area of the ECG report further includes an MD confirmation button, wherein the MD confirmation button indicates whether a clinician confirmed the set of outcomes.

7. The system of claim 1, wherein the ECG report includes a clinical decision support box, wherein the clinical decision support box includes a condition conclusion.

8. The system of claim 1, wherein the ECG report includes an ECG serial box that includes a set of ECG serial data and a serial test indication.

9. The system of claim 8, wherein the ECG results box provides a serial status for the ECG serial box.

10. The system of claim 1, wherein the EMR data comprises a patient cardiac history.

11. In a computer system for electrocardiogram (ECG) analysis having a graphical user interface (GUI) including a display and an input device as a method for providing electrocardiogram (ECG) data in an electronic medical record (EMR), the method comprising:
receiving ECG data and electronic medical record (EMR) data;
correlating the EMR data with the ECG data;
analyzing the ECG data and EMR data; and
conveying an ECG report in the GUI, wherein the ECG report includes the EMR data and the ECG data.

12. The computer system of claim 11, wherein the ECG report in the GUI further includes a test indication, wherein the test includes a time and a reason for an ECG test.

13. The computer system of claim 11, wherein the ECG report in the GUI further includes a clinical findings area.

14. The computer system of claim 13, wherein the clinical findings area of the ECG report in the GUI further includes an ECG data values section, further wherein the ECG data values provides an indication of any abnormal values in the ECG data.

15. The computer system of claim 13, wherein the clinical findings area of the ECG report in the GUI further includes an ECG results box, further wherein the ECG results box provide a standard ECG interpretation from the ECG data.

16. The computer system of claim 15, wherein the clinical findings area of the ECG report in the GUI further includes an MD confirmation button, wherein the MD confirmation button indicates whether a clinician confirmed the set of outcomes.

17. The computer system of claim 11, wherein the ECG report in the GUI further includes a clinical decision support box, wherein the clinical decision support box includes a condition conclusion.

18. The computer system of claim 11, wherein the ECG report in the GUI includes an ECG serial box that includes a set of ECG serial data and a serial test indication.

19. The computer system of claim 18, wherein the ECG results box in the GUI provides a serial status for the ECG serial box.

20. A system for electrocardiogram (ECG) analysis, comprising:
an ECG monitor configured to collect ECG data from a patient;
an electronic medical record (EMR) database configured to store EMR data for the patient;
a processor configured to receive the EMR data, and retrieve the ECG data from the ECG monitor through a portal, wherein the EMR data is correlated with the ECG data, and the processor is further configured to analyze the ECG data and EMR data; and
a display operatively connected to the processor and configured to convey an ECG report in a graphical user interface, wherein the ECG report includes the EMR data and the ECG data.
